(19) United States
(12) Patent Application Publication
(10) Pub. No.: US 2005/0159768 A1
(43) Pub. Date: Jul. 21, 2005

(54) Lancing device

(75) Inventors: David K. Boehm, Lauderhill, FL (US); Cameron Scott Casterline, Pembroke Pines, FL (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: Home Diagnostics, Inc., Fort Lauderdale, FL (US)

(21) Appl. No.: 10/757,776

(22) Filed: Jan. 15, 2004

(51) Int. Cl. A61B 17/32

(52) U.S. Cl. 606/182

(57) ABSTRACT

An apparatus and method for lancing a surface provides an adjustable nozzle assembly that includes an interior nozzle, a collar, and an exterior nozzle with a surface that contacts the surface to be lanced. The exterior nozzle can then rotate relative to the interior nozzle and, thereby, vary the lancing depth of a lancet. The apparatus and method for lancing a surface also provides for a rearward body assembly that includes an interior tube, a lancet holder, an internal compression spring, a retainer, a rearward body, and an external compression spring. Longitudinal movement of the rearward body away from the interior tube can compress the interior compression spring and can, thereby, spring load the lancing device. The apparatus and method for assembling the adjustable nozzle assembly of the lancing device provides for an interior nozzle with an assembly groove and a ramped groove separated by a raised boss. The apparatus and method for assembling also provides for a collar with a collar pin for sliding in the assembly groove, over the raised boss, and into the ramped groove.

### BACKGROUND

1. Field of the Invention

The present invention relates to lancing devices and, more particularly, to apparatuses and methods for lancing and assembling lancing devices.

2. Description of Related Art

Many people have a need to regularly monitor their blood. Diabetics, for example, need to regularly monitor their blood glucose levels. Lancing devices, generally, offer a relatively pain-free, quick, and sterile means to prick a person's skin to obtain a drop of blood. The blood may then be collected and/or tested, in a blood glucose meter, for example.

To obtain a drop of blood, a spring-loaded lancing device can be placed in contact with a person's skin. The loaded spring can then be released, and the lancing device can propel a needle-tipped lancet through a hole in a nozzle of the lancing device into the skin. The needle can then penetrate the skin for only a fraction of a second before being retracted back into the lancing device.

The depth that the needle penetrates the skin can be considered the lancing depth. The lancing depth can then generally be equal to the furthest length that the needle extends from the nozzle of the lancing device as the needle penetrates the skin.

The lancing depth can be controlled, for example, by using a particular length needle. Another, more convenient, way to control the lancing depth is by rotating the nozzle of the lancing device to adjust the relative distance between the nozzle and the tip of the lancet's needle. For instance, for a shallower lancing depth, the nozzle can be moved closer to the needle tip at the needle tip's furthest distance from the nozzle. For a deeper lancing depth, the nozzle can be moved further away from the needle tip at the needle tip's furthest distance from the nozzle.

A lancing depth that can be conveniently adjusted has many advantages. Different fingers can have different skin thicknesses, for example. Different areas of the body might also have different skin thicknesses. Adjusting a lancing device to the appropriate lancing depth can therefore minimize the pain of lancing too deeply into the skin and can help ensure that the lancet needle penetrates the skin a sufficient depth to obtain a drop of blood.

Lancing devices, generally, can be beneficial for many reasons. For example, lancing devices offer a less painful way to prick skin to obtain a drop of blood. Pain is minimized because the lancet's needle is in the skin for only a fraction of a second. Lancing devices can also minimize the emotional trauma of pricking skin. When using a lancing device one can simply, for example, press a button to lance one's skin, instead of somehow holding a needle and directly pushing the needle into the skin. Lancing devices can also hide the lancet needle within an enclosure, thereby allowing the person being stuck to avoid looking at the needle. In a clinical setting, lancing devices can also allow a nurse or other caregiver to quickly and relatively painlessly prick a patient's skin to obtain a drop of blood.

Accordingly, there is a need to provide convenient and reliable lancing devices, methods of lancing, and methods of assembling lancing devices.

### SUMMARY

Exemplary embodiments include a simple to operate handheld device with a “pen like” ergonomic styling. One embodiment has five adjustable lancing depth settings. The embodiments can be used for normal finger tip lancing or for alternate site testing, and can use standard (universal), “off the shelf” lancets. The lancing device can also be constructed of various injection molded plastic parts that can “snap-fit” or “pressure-fit” together.

In a first embodiment, the present invention provides an adjustable nozzle assembly through which a lancet can be propelled by a lancing device into a lancing surface. The adjustable nozzle assembly includes an interior nozzle comprising a ramped groove and a lancet wall; a collar comprising a collar pin that engages the ramped groove and slides relative to the ramped groove, the collar being adapted to rotate relative to the interior nozzle; and an exterior nozzle comprising a contact surface that extends beyond the lancet wall of the interior nozzle to contact the lancing surface, the exterior nozzle engaging the collar and being adapted to rotate relative to the interior nozzle. The ramped groove is sloped such that as the exterior nozzle rotates relative to the interior nozzle, the distance that the contact surface extends beyond the lancet wall changes by an amount that corresponds to the slope of the ramped groove. In this and other embodiments, the collar can further comprise a detent, and the interior nozzle can further comprise a plurality of adjustment notches that can engage the detent.

In a second embodiment, the present invention provides a rearward body assembly of a lancing device that can propel a lancet into a lancing surface. The rearward body assembly includes a lancet holder comprising one or more retaining features and one or more spring surfaces; an interior tube comprising an open end and a slotted end through which the one or more retaining features extend, the interior tube being adapted to slidably engage the lancet holder; an internal compression spring comprising a first end and a second end, the first end of the internal compression spring being adapted to act on the slotted end of the interior tube and the second end of the internal compression spring being adapted to act on the one or more spring surfaces of the lancet holder; a retainer comprising a slotted surface through which the one or more retaining features extend; a rearward body, the rearward body engaging the retainer; and an external compression spring comprising a first end and a second end, the first end comprising a reduced coil diameter that engages the one or more retaining features of the lancet holder, the first end of the external compression spring being adapted to act on the lancet holder, and the second end of the external compression spring being adapted to act on the slotted surface of the retainer. Longitudinal movement of the rearward body away from the interior tube compresses the interior compression spring.

In a third embodiment, the present invention provides that the rearward body assembly of the second embodiment can releasably engage the adjustable nozzle assembly of the first embodiment.

In a fourth embodiment, the present invention provides a method of assembling an adjustable nozzle assembly of a lancing device. The method of the fourth embodiment includes providing an interior nozzle comprising an assembly groove in communication with a ramped groove, the assembly groove being separated from the ramped groove by a raised boss; providing a collar with a collar pin; and attaching the collar to the interior nozzle by sliding the collar pin in the assembly groove, over the raised boss, and into the ramped groove. In this and other embodiments, the collar can further comprise a detent, and the interior nozzle can further comprise a plurality of adjustment notches that can engage the detent.

In a fifth embodiment, the present invention provides a method of adjusting a lancing depth of a nozzle assembly in a lancing device, the nozzle assembly comprising an exterior nozzle, an interior nozzle comprising a ramped groove, and a collar engaging the exterior nozzle and comprising a collar pin that can slidably engage the ramped groove. The method includes rotating the exterior nozzle and the collar relative to the interior nozzle to slide the collar pin in the ramped groove of the interior nozzle to adjust the lancing depth.

Other embodiments are possible as well, and a variety of alternatives will become apparent to those skilled in the art upon review of the following detailed description.

### BRIEF DESCRIPTION OF THE DRAWINGS

Exemplary embodiments of the present invention are described herein with reference to the drawings, in which:

FIG. 1 is a perspective view of an exemplary lancing device;

FIG. 2 is a perspective view of an exemplary lancet;

FIG. 3 is an exploded perspective view of several components of the lancing device, including a finger cover, a trigger, and an interior tube;

FIG. 4 is a cross-sectional perspective view of several components of the lancing device, including the components of FIG. 3 and a lancet holder and an internal spring;

FIG. 4A is a perspective view of a detail of the finger cover engaging the interior tube;

FIG. 5 is a perspective view of the components of FIG. 4 engaging a retainer and an external spring;

FIG. 6 is an exploded perspective view of the components of FIG. 5 engaging a rearward body;

FIG. 7 is a perspective view of a rearward body assembly, including the finger cover, the trigger, the interior tube, and the lancet holder;

FIG. 8 is an exploded perspective view of an exterior nozzle and a collar of the lancing device;

FIG. 8A is a perspective view of a detail of the exterior nozzle and the collar;

FIG. 9 is an exploded perspective view of the components of FIG. 8 and an interior nozzle;

FIG. 9A is a perspective view of an alternative collar embodiment;

FIG. 10 is an exploded perspective view of the components of FIG. 8 and the interior nozzle;

FIG. 11 is a perspective view of the nozzle assembly, including the exterior nozzle and the interior nozzle;

FIG. 12 is a perspective view of the nozzle assembly and the rearward body assembly engaging the lancet;

FIG. 13 is a perspective view of an exemplary exterior nozzle configuration;

FIG. 14 is a perspective view of an exemplary exterior nozzle configuration; and

FIG. 15 is a perspective view of an exemplary exterior nozzle configuration.

### DETAILED DESCRIPTION OF THE EXEMPLARY EMBODIMENTS

1. Exemplary Lancing Device Configurations

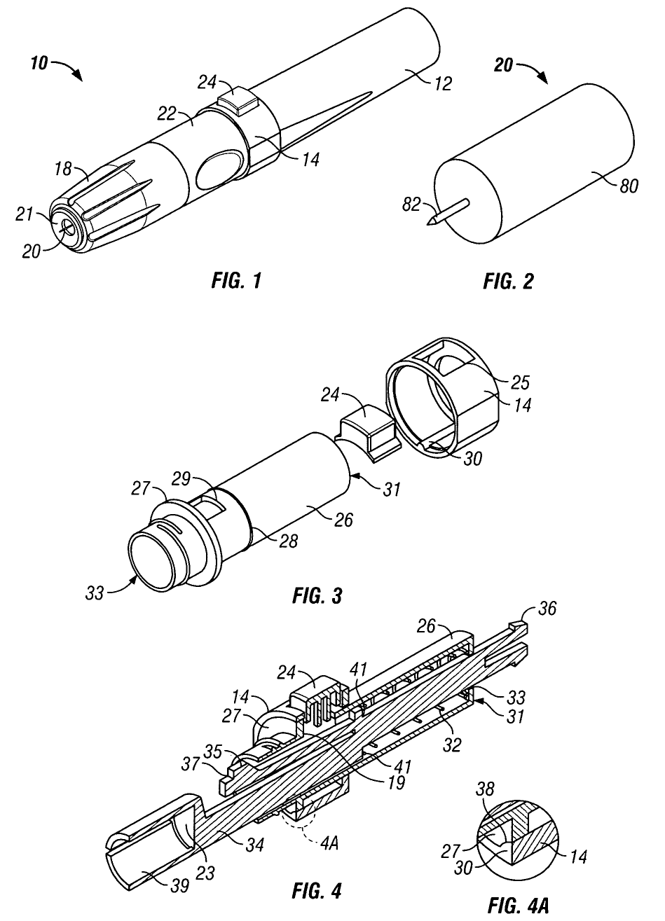

FIG. 1 depicts an exemplary lancing device 10. The lancing device 10 can include a rearward body 12, a finger cover 14, an exterior nozzle 18 with a contact surface 21, an interior nozzle 22, and a trigger 24. The components of the lancing device 10 can be assembled to spring load and propel a lancet 20. Upon being propelled, the tip of the lancet 20 can extend beyond the contact surface 21 a particular, adjustable lancing depth. In some embodiments, the lancet 20 is removable or replaceable. For instance, in many applications it is advantageous to have a disposable, single-use lancet 20.

FIG. 2 depicts an exemplary lancet 20 for use in the lancing device 10. As shown in FIG. 2, the lancet 20 can comprise a needle section 82 and a barrel section 80. The lengths and diameters of each of the needle section 82 and the barrel section 80 can vary. Although the barrel section 80 is shown as smooth, the barrel section 80 can also be contoured.

FIG. 3 depicts an exploded view of several components of the lancing device 10, in particular, an interior tube 26, the finger cover 14, and the trigger 24. As shown in FIG. 3, the interior tube 26 has a distal end 31 and a proximal end 33, and can include a snap ring 28, a raised ring 27, and an interior tube opening 29. The finger cover 14 can comprise a finger cover alignment feature 30, which might be an elongated protrusion on the inner surface of the interior tube 26, and a trigger opening 25. Once assembled, the trigger 24 can extend outwardly though the trigger opening 25 and can extend inwardly through the interior tube opening 29.

FIG. 4 depicts a cross-sectional perspective view of several components of the lancing device 10, including the components of FIG. 3, an internal spring 32, and a lancet holder 34. In an exemplary embodiment, the internal spring 32 is a compression spring that can act against the distal end 31 of the interior tube 26 and against the lancet holder 34 to propel the lancet 20.

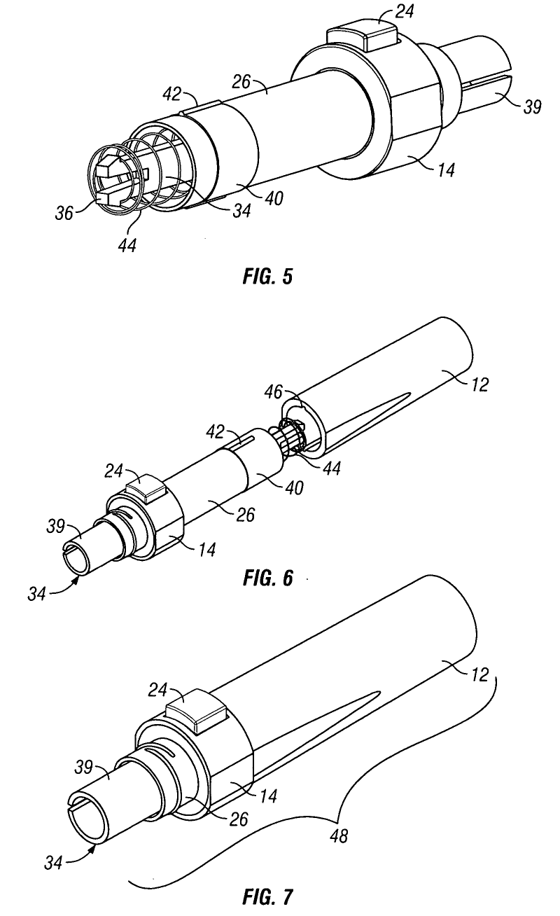

In an exemplary embodiment, the lancet holder 34 includes a plurality of components and serves a plurality of functions. The lancet holder 34 can comprise a barrel holder 39 and a seat 23, both of which can hold and help propel the lancet 20. The lancet holder 34 can also comprise one or more internal spring surfaces 41 against which the internal spring 32 can act to propel the lancet 20. Further, the lancet holder 34 can comprise one or more retaining features 36 that can extend through a slot 33 of the interior tube 26 and can help retain an external spring 44 and a retainer 40 (both shown in FIG. 5). In addition, the lancet holder can also comprise a cantilevered trigger extension 35, which can be biased such that it can spring load the trigger 24.

To spring load the trigger 24 and prepare the lancing device 10 for use, the trigger extension 35 can be aligned with the interior tube opening 29 by compressing the internal spring 32. Once sufficiently aligned, the bias of the trigger extension 35 can cause the trigger extension 35 to extend toward the opening 29 and engage the trigger 24. A trigger extension notch 37 on the trigger extension 35 can then engage a corner 19 of the interior tube 26. The trigger extension 35 can thereby oppose longitudinal movement of the lancet holder 34 and can oppose the longitudinal force caused by the compressed internal spring 32.

There is thus a “loaded” position of the lancing device 10 in which the internal spring 32 is compressed and the engagement of the notch 37 with the interior tube 26 opposes the release of the compressed internal spring 32. In the loaded position, the trigger extension 35 engages the trigger 24 such that, in an exemplary embodiment, the trigger is somewhat raised from the surface of the finger cover 14 and offers some resistance to movement. To propel the lancet 20, a user can actuate the trigger 24 to release the engagement of the notch 37 with the interior tube 24 and to thereby release the internal spring 32. The internal spring 32 can then move the lancet holder 34 longitudinally to propel the lancet 20.

In assembly, both the finger cover alignment feature 30 and the snap ring 28 can help align the finger cover 14 with other components of the lancing device 10. As shown in FIG. 4A, the finger cover alignment feature 30 can help align the finger cover 14 along the periphery of the interior tube 26 by engaging a notch 38 in the raised ring 27. In an exemplary embodiment, the snap ring 28 can help align the finger cover 14 along the longitudinal axis of the interior tube 26 by engaging an edge of the finger cover 14.

FIG. 5 depicts the components of FIG. 4 engaging a retainer 40 and an external spring 44. The external spring 44 can engage the retaining features 36 of the lancet holder 34 and an end of the retainer 40. In an exemplary embodiment, the diameter of the external spring 44 is reduced near where the external spring 44 engages the retaining features 36, and, in an exemplary embodiment, the retaining features 36 can include an aggressive undercut to facilitate engagement with the external spring 44. Advantageously, the reduced external spring diameter allows the external spring 44 to act directly on the lancet holder. The reduced spring diameter can thus eliminate the need for an additional component for the external spring 44 to act on, such as a slotted disc connected to the retaining features 36 of the lancet holder 34. In an exemplary embodiment, the retainer 40 can include one or more retainer alignment features 42.

FIG. 6 depicts an exploded view of the components of FIG. 5 engaging the rearward body 12. In an exemplary embodiment, the rearward body 12 can include one or more grooves 46. The grooves 46 can engage the retainer alignment features 42 and can thereby help align the rearward body 12 along the circumference of the lancing device 10. Advantageously, after the rearward body 12 is assembled with the retainer 40, the grooves 46 and the retainer alignment features 42 can also oppose rotation of the rearward body along its longitudinal axis. The rearward body 12 might also include a snap ring feature (not shown) that can help align the rearward body 12 along the longitudinal axis of the lancing device 10 and that can help ensure that the rearward body 12 is fully engaged with the lancing device 10.

FIG. 7 depicts a rearward body assembly 48, which includes the rearward body 12 assembled with the finger cover 14, the trigger 24, the interior tube 26, and the lancet holder 34. A user can “load” the rearward body assembly 48 by longitudinally pulling the rearward body 12 away from the finger cover 14. Because the rearward body 12 is coupled to the retainer 40, and because the retainer 40 is coupled to the lancet holder 34 (via the external spring 44), the movement of the rearward body 12 will compress the internal spring 32 and will longitudinally move the lancet holder 34 relative to the interior tube 26. Once the trigger extension 35 is sufficiently aligned with the interior tube opening 29, the trigger extension 35 can engage the trigger 24, and the notch 37 can engage the interior tube 26 to oppose the release of the compressed spring 32. The user can then release the rearward body 12 and the external spring 44 will return the rearward body 12 to the position depicted in FIG. 7. Once the rearward body assembly 48 is loaded, the user can then actuate the trigger 24 to release the lancet holder 34.

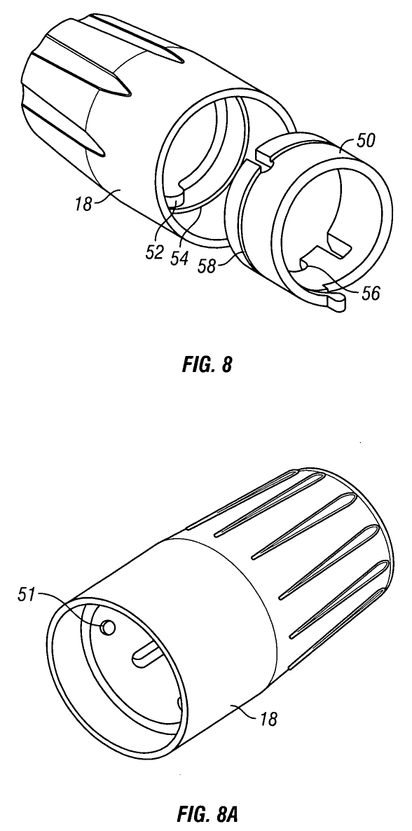

FIG. 8 depicts an exploded view of the exterior nozzle 18 and a collar 50. In an exemplary embodiment, the collar 50 comprises a retaining groove 58 and one or more collar alignment features 56, and the exterior nozzle 18 comprises a retaining ring 54 and one or more exterior nozzle alignment features 52. The collar 50 can then be inserted into the exterior nozzle 18, and the retaining groove 58 can engage the retaining ring 54 to help align and retain the collar 50 along the longitudinal axis of the exterior nozzle 18. Further, the collar alignment features 56 can engage the exterior nozzle alignment features 52 to help align and retain the collar 50 along the interior circumference of the exterior nozzle 18.

The interior surface of the collar 50 can also comprise a collar pin 51, which is depicted in FIG. 8A. As shown in FIG. 8A, the collar pin 51 can extend from the interior surface of the collar 50.

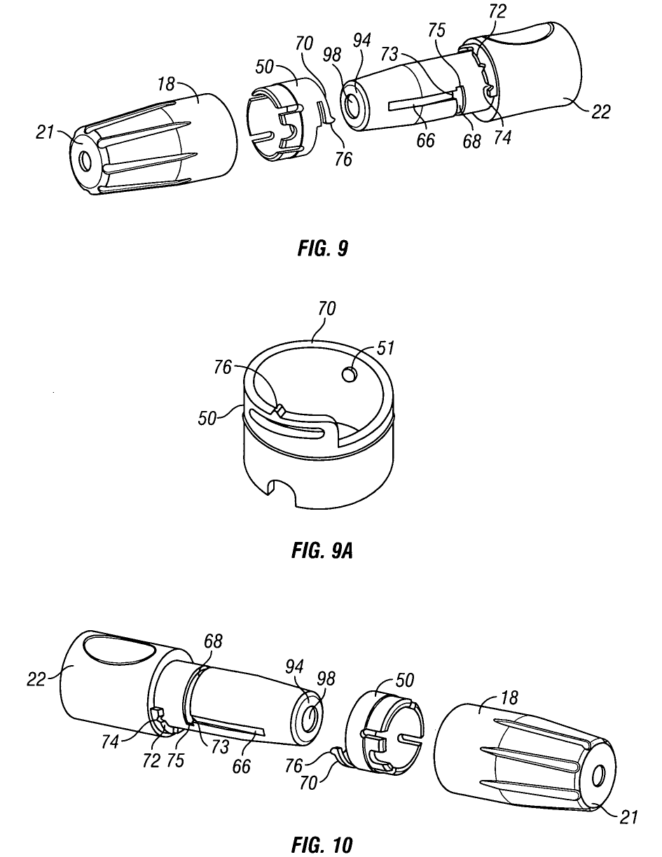

FIGS. 9 and 10 depict exploded views (from different perspectives) of the components of FIG. 8 and the interior nozzle 22. As shown in FIGS. 9 and 10, the interior nozzle 22 can comprise an assembly groove 66 and a ramped groove 68. The collar 50 can then engage the interior nozzle 22 via the collar pin 51 (shown in FIG. 8A), which can engage the assembly groove 66 and the ramped groove 68 of the interior nozzle 22.

In an exemplary embodiment, to assemble the collar 50 and the interior nozzle 22, one can slide the collar pin 51 through the assembly groove 66 to the ramped groove 68. Near where the assembly groove 66 meets the ramped groove 68, the interior nozzle 22 can comprise a raised boss 73. In assembly, the collar pin 51 can be slid in the assembly groove 66 and can then snap over the raised boss 73 into the ramped groove 68.

Once assembled, the raised boss 73 creates a tortuous path that can make disassembly of the collar 50 (and hence the exterior nozzle 18) from the interior nozzle 22 difficult. The ramped groove 68 can retain the collar pin 51 and can allow the collar to rotate about the periphery of the interior nozzle 22. The interior nozzle 22 can also comprise an over rotation groove 75 that can retain the collar pin 51 when the collar is fully rotated and can help prevent disassembly of the collar 50 (and hence the exterior nozzle 18) from the interior nozzle 22 when the collar 50 and the exterior nozzle 18 are fully rotated.

As shown in FIGS. 9 and 10, the interior nozzle 22 can also comprise an interior nozzle mating ramp 72, which can include a plurality of adjustment notches 74 (in a preferred embodiment, for example, there are five adjustment notches 74). The collar 50, in turn, can comprise a collar mating ramp 70, which can include a detent 76. The detent 76 can form a biased, flexible portion of the collar mating ramp 70, to allow the detent to engage and disengage the adjustment notches 74. In an exemplary embodiment, the detent 76 forms a cantilevered portion of the collar mating ramp 70. In an alternative embodiment depicted in FIG. 9A, the detent 76 forms a slotted portion of the collar mating ramp 70. Other examples are possible as well.

The interior nozzle mating ramp 72, the collar mating ramp 70, and the ramped groove 68, can each be sloped relative to a plane perpendicular to the longitudinal axis of the lancing device 10. Further, in an exemplary embodiment, the slopes of each of the interior nozzle mating ramp 72, the collar mating ramp 70, and the ramped groove 68 should be approximately the same or similar. Once assembled, the collar pin 51 can then slide in the ramped groove 68 and the collar mating ramp 70 and the detent 76 can rotate along the interior nozzle mating ramp 72.

As shown in FIGS. 9 and 10, the interior nozzle 22 can comprise a lancet wall 94 with a lancet wall opening 98. When a lancet 20 is propelled by the lancing device 10, the lancet wall opening 98 can provide an opening through which the needle section 82 can extend beyond the lancet wall 94 toward the surface to be lanced. The lancet wall 94, in turn, can engage the barrel section 80 of the lancet 20 and can act to stop the movement of a propelled lancet 20.

As discussed above, in an assembled lancing device 10, the exterior nozzle 18 engages the collar 50. Thus, the collar pin 51, the collar 50, and the exterior nozzle 18 rotate together about the periphery of the interior nozzle 22. By sliding in the ramped groove 68, the collar pin 51 can act to vary the distance that the contact surface 21 of the exterior nozzle 18 extends from the lancet wall 94, and, hence, can act to vary the distance that the lancet 20 extends from the contact surface 21. Thus, by rotating the exterior nozzle 18 relative to other components of the lancing device 10, one can vary the lancing depth according to the slope of the ramped groove 68.

In an exemplary embodiment, particular lancing depth positions can be defined by the adjustment notches 74 along the slope of the interior nozzle mating ramp 72. Then as the exterior nozzle 18 is rotated, the detent 76 of the collar mating ramp 70 can engage the various adjustment notches 74. The engagement between the detent 76 and each adjustment notch 74 can help define a particular lancing depth for the lancet 20. An indication of the location and the corresponding, relative lancing depth represented by each of the adjustment notches 74 can be marked on the outside surface of the lancing device 10.

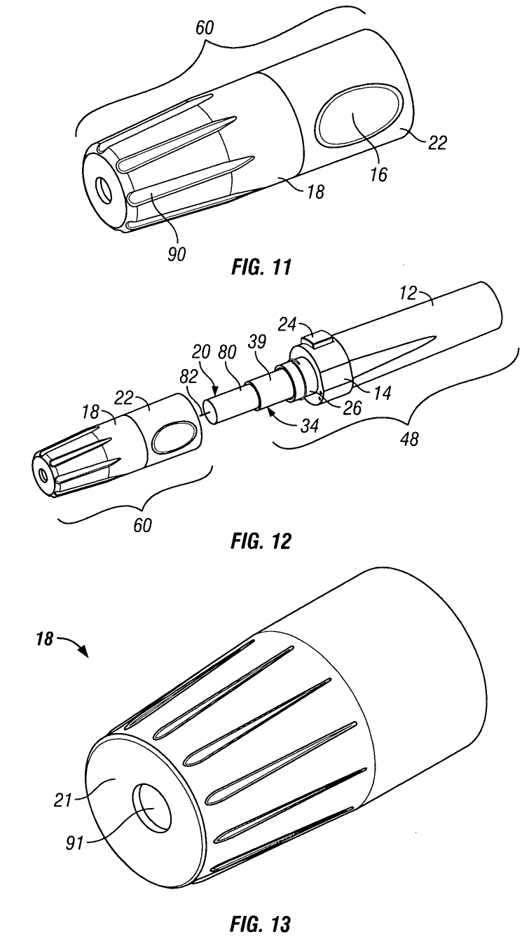

FIG. 11 depicts a nozzle assembly 60, which includes the exterior nozzle 18, the collar 50 (shown in FIGS. 9 and 10), and the interior nozzle 22. As discussed above, a user can adjust the lancing depth of the lancet 20 by rotating the exterior nozzle 18 relative to the interior nozzle 22. To facilitate the rotation of the exterior nozzle 18, the exterior nozzle 18 can comprise a plurality of grip features 90, and the interior nozzle 22 can comprise one or more finger grips 16. One can then hold the interior nozzle 22 at the finger grips 16 and rotate the exterior nozzle 18 using the grip features 90. In an exemplary embodiment, as the exterior nozzle 18 is rotated, the detent 76 can “click” into the various adjustment notches 76 (shown in FIGS. 9 and 10) on the interior nozzle 22.

FIG. 12 depicts a perspective view of the nozzle assembly 60 and the rearward body assembly 48 engaging the lancet 20. As shown in FIG. 12, the barrel holder 39 of the lancet holder 34 can engage the barrel section 80 of the lancet 20. The lancet 20 can then rest in the seat 23 (shown in FIG. 4) of the lancet holder 34. In an exemplary embodiment, the interior diameter of the barrel holder 39 can approximately equal the outside diameter of the barrel section 80.

As also shown in FIG. 12, in an exemplary embodiment, the nozzle assembly 60 can be releasably coupled to the rearward body assembly 48 via a threaded connection, for example. Other methods of attachment, such as a snap-fit, quick-release, or sliding coupling, are possible as well. In any case, the advantages of such a releasable coupling include allowing a user to disengage the nozzle assembly 60 from the rearward body assembly 48 to insert or remove a lancet 20 from the lancet holder 34.

FIGS. 13, 14, and 15 depict exemplary exterior nozzle 18 configurations. Each exterior nozzle 18 includes the contact surface 21, which can contact the surface that the lancet 20 will lance. The contact surface 21, in turn, will comprise a lancet opening 91, through which the needle section 82 of a lancet 20 can extend to lance a surface, such as a person's skin. In general, it is advantageous for the lancing device 10 to have a contact surface 21 that is shaped to encourage the flow of blood to the point where the lancet 20 lances the skin.

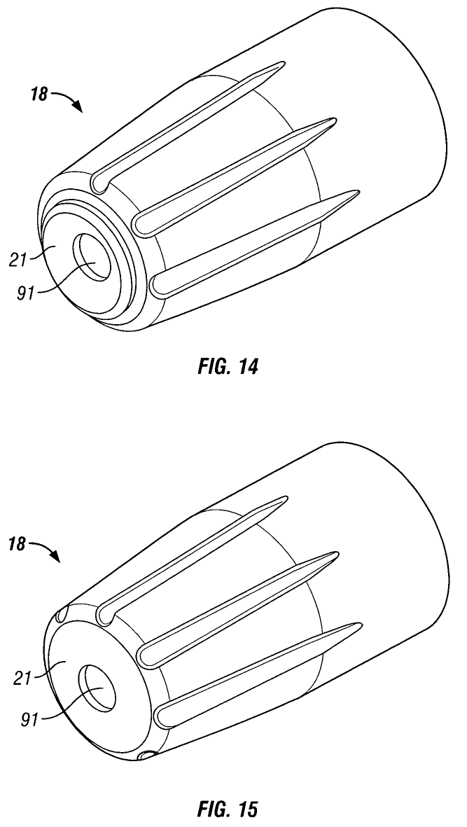

The exterior nozzles depicted in FIGS. 13, 14, and 15 differ in that the contact surface 21 of each exterior nozzle 18 has different features. FIG. 13 depicts the exterior nozzle 18 with a concave contact surface 21. Although the contact surface 21 shown in FIG. 13 is smooth, the surface 21 can also have a contoured topography. FIG. 14 depicts the exterior nozzle 18 with a raised-ring, contoured contact surface 21. And FIG. 15 depicts the exterior nozzle 18 with a planar contact surface 21. Other examples, such as segmented contact surfaces, for example, are possible as well.

2. Exemplary Operation

With reference to the above discussed figures, operation of the lancing device 10 can include several steps. These steps might include insertion of a lancet 20 into the lancing device 10, selection of a lancing depth, spring loading the lancing device 10, and actuating the trigger 24 to lance a surface, such as a person's skin.

In particular, to insert a lancet 20 into the lancing device 10, a user can disengage the nozzle assembly 60 from the rearward body assembly 48 to expose the barrel holder 39 of the lancet holder 34. The user can then insert the barrel section 80 of the lancet 20 into the barrel holder 39 until the lancet 20 rests against the seat 23 of the lancet holder 34. As necessary, the user can remove any protective or safety cover on the lancet 20 to expose the needle section 82 of the lancet 20. The user can then re-engage the nozzle assembly 60 with the rearward body assembly 48.

Next the user can select a particular lancing depth of the lancet by rotating the exterior nozzle 18. The exterior nozzle 18 can be marked with identifications of available lancing depths that correspond with the adjustment notches 74. As the user rotates the exterior nozzle 18, the detent 76 can click into the adjustment notches 74, indicating to the user that a particular lancing depth has been selected.

The user can then spring load the lancing device 10 by pulling the rearward body 12 along the longitudinal axis of the lancing device 10. To hold the lancing device while pulling the rearward body 12, the user can grip another component, such as the finger cover 14 or the interior nozzle 22. In an exemplary embodiment, once the user has pulled the rearward body 12 a sufficient amount, the user can see and/or hear the trigger extension 35 engaging the trigger 24 and/or the trigger extension notch 37 engaging the interior tube 26. The user can then release the rearward body 12, which will return to its original position via the external spring 44 acting on the retainer 40.

To use the lancing device 10, the user can place the contact surface 21 of the external nozzle 18 on the surface to be lanced, such as a person's skin. The user can then actuate the trigger 24 and the internal spring 32 will propel the lancet holder 34 and, hence, the lancet 20, toward the lancing surface. The needle section 82 will also be propelled through the lancet wall opening 98, through the lancet opening 91, and into the lancing surface, up to the lancing depth. Blood can then be collected from the lanced surface and tested.

### 3. CONCLUSION

Exemplary embodiments of the present invention have been described above. Those skilled in the art will understand, however, that changes and modifications may be made to these embodiments without departing from the true scope and spirit of the invention, which is defined by the claims.

1. An adjustable nozzle assembly through which a lancet can be propelled by a lancing device into a lancing surface, the adjustable nozzle assembly comprising:
an interior nozzle comprising a ramped groove and a lancet wall;
a collar comprising a collar pin that engages the ramped groove and slides relative to the ramped groove, the collar being adapted to rotate relative to the interior nozzle; and
an exterior nozzle comprising a contact surface that extends beyond the lancet wall of the interior nozzle to contact the lancing surface, the exterior nozzle engaging the collar and being adapted to rotate relative to the interior nozzle; and
wherein the ramped groove is sloped such that as the exterior nozzle rotates relative to the interior nozzle, the distance that the contact surface extends beyond the lancet wall changes by an amount that corresponds to the slope of the ramped groove.

2. The nozzle assembly of claim 1, wherein the collar further comprises a cantilevered detent and wherein the interior nozzle further comprises a plurality of adjustment notches that can engage the cantilevered detent.

3. The nozzle assembly of claim 1, wherein:
the collar further comprises a sloped collar ramp, the sloped collar ramp comprising a detent; and
the interior nozzle further comprises a sloped interior nozzle ramp, the sloped interior nozzle ramp comprising a plurality of adjustment notches that can engage the detent; and
wherein the slope of the collar ramp, the slope of the interior nozzle ramp, and the slope of the ramped groove are approximately equal.

4. The nozzle assembly of claim 3, wherein the detent forms a cantilevered portion of the collar ramp.

5. The nozzle assembly of claim 3, wherein the detent forms a slotted portion of the collar ramp.

6. The nozzle assembly of claim 1, wherein the interior nozzle further comprises an assembly groove, one end of the assembly groove being in proximity to one end of the ramped groove, the assembly groove comprising a raised boss that can oppose the collar pin sliding from the ramped groove to the assembly groove.

7. The nozzle assembly of claim 1, wherein the collar further comprises one or more collar alignment features, and the exterior nozzle further comprises one or more exterior nozzle alignment features that can engage the one or more collar alignment features.

8. The nozzle assembly of claim 1, wherein the contact surface is concave.

9. The nozzle assembly of claim 1, wherein the ramped groove comprises an over-rotation groove.

10. A rearward body assembly of a lancing device that can propel a lancet into a lancing surface, the rearward body assembly comprising:
a lancet holder comprising one or more retaining features and one or more spring surfaces;
an interior tube comprising an open end and a slotted end through which the one or more retaining features extend, the interior tube being adapted to slidably engage the lancet holder;
an internal compression spring comprising a first end and a second end, the first end of the internal compression spring being adapted to act on the slotted end of the interior tube and the second end of the internal compression spring being adapted to act on the one or more spring surfaces of the lancet holder;
a retainer comprising a slotted surface through which the one or more retaining features extend;
a rearward body, the rearward body engaging the retainer; and
an external compression spring comprising a first end and a second end, the first end comprising a reduced coil diameter that engages the one or more retaining features of the lancet holder, the first end of the external compression spring being adapted to act on the lancet holder and the second end of the external compression spring being adapted to act on the slotted surface of the retainer; and
wherein longitudinal movement of the rearward body away from the interior tube compresses the interior compression spring.

11. The lancing device of claim 10, wherein the lancet holder further comprises a trigger extension, the trigger extension being adapted to engage both a trigger and the interior tube to load the lancing device and to oppose the force of the compression spring until the trigger is actuated.

12. The lancing device of claim 10, wherein the retainer further comprises one or more retainer alignment features, and the rearward body further comprises one or more rearward body alignment features that can engage the one or more retainer alignment features.

13. A lancing device that can propel a lancet into a lancing surface, the lancing device comprising:
(i) an adjustable nozzle assembly through which the lancet can be propelled into a lancing surface, the adjustable nozzle assembly comprising:
an interior nozzle comprising a ramped groove and a lancet wall;
a collar comprising a collar pin that engages the ramped groove and slides relative to the ramped groove, the collar being adapted to rotate relative to the interior nozzle; and
an exterior nozzle comprising a contact surface that extends beyond the lancet wall of the interior nozzle to contact the lancing surface, the exterior nozzle engaging the collar and being adapted to rotate relative to the interior nozzle; and
wherein the ramped groove is sloped such that as the exterior nozzle rotates relative to the interior nozzle, the distance that the contact surface extends beyond the lancet wall changes by an amount that corresponds to the slope of the ramped groove; and
(ii) a rearward body assembly, the rearward body assembly comprising:
a lancet holder comprising one or more retaining features and one or more spring surfaces;
an interior tube comprising an open end and a slotted end through which the one or more retaining features extend, the interior tube being adapted to slidably engage the lancet holder;
an internal compression spring comprising a first end and a second end, the first end of the internal compression spring being adapted to act on the slotted end of the interior tube and the second end of the internal compression spring being adapted to act on the one or more spring surfaces of the lancet holder;
a retainer comprising a slotted surface through which the one or more retaining features extend;
a rearward body, the rearward body engaging the retainer; and
an external compression spring comprising a first end and a second end, the first end comprising a reduced coil diameter that engages the one or more retaining features of the lancet holder, the first end of the external compression spring being adapted to act on the lancet holder, and the second end of the external compression spring being adapted to act on the slotted surface of the retainer; and
wherein longitudinal movement of the rearward body away from the interior tube compresses the interior compression spring; and
wherein the rearward body assembly can releasably engage the adjustable nozzle assembly.

14. The lancing device of claim 13, wherein the collar further comprises a cantilevered detent and wherein the interior nozzle further comprises a plurality of adjustment notches that can engage the cantilevered detent.

15. The lancing device of claim 13, wherein:
the collar further comprises a sloped collar ramp, the sloped collar ramp comprising a cantilevered detent; and
the interior nozzle further comprises a sloped interior nozzle ramp, the sloped interior nozzle ramp comprising a plurality of adjustment notches that can engage the cantilevered detent; and
wherein the slope of the collar ramp, the slope of the interior nozzle ramp, and the slope of the ramped groove are approximately equal.

16. The lancing device of claim 15, wherein the detent forms a cantilevered portion of the collar ramp.

17. The lancing device of claim 15, wherein the detent forms a slotted portion of the collar ramp.

18. The lancing device of claim 13, wherein the interior nozzle further comprises an assembly groove, one end of the assembly groove being in proximity to one end of the ramped groove, the assembly groove comprising a raised boss that can oppose the collar pin sliding from the ramped groove to the assembly groove.

19. The lancing device of claim 13, wherein the collar further comprises one or more collar alignment features, and the exterior nozzle further comprises one or more exterior nozzle alignment features that can engage the one or more collar alignment features.

20. The lancing device of claim 13, wherein the contact surface is concave.

21. The lancing device of claim 13, wherein the ramped groove comprises an over-rotation groove.

22. The lancing device of claim 13, wherein the lancet holder further comprises a trigger extension, the trigger extension being adapted to engage both a trigger and the interior tube to load the lancing device and to oppose the force of the compression spring until the trigger is actuated.

23. The lancing device of claim 13, wherein the retainer further comprises one or more retainer alignment features, and the rearward body further comprises one or more rearward body alignment features that can engage the one or more retainer alignment features.

24. A method of assembling an adjustable nozzle assembly of a lancing device, the method comprising:
providing an interior nozzle comprising an assembly groove in communication with a ramped groove, the assembly groove being separated from the ramped groove by a raised boss;
providing a collar with a collar pin; and
attaching the collar to the interior nozzle by sliding the collar pin in the assembly groove, over the raised boss, and into the ramped groove.

25. The method of claim 24, wherein the collar further comprises a cantilevered detent and wherein the interior nozzle further comprises a plurality of adjustment notches that can engage the cantilevered detent.

26. The method of claim 24, wherein:
the collar further comprises a sloped collar ramp, the sloped collar ramp comprising a cantilevered detent; and
the interior nozzle further comprises a sloped interior nozzle ramp, the sloped interior nozzle ramp comprising a plurality of adjustment notches that can engage the cantilevered detent; and
wherein the slope of the collar ramp, the slope of the interior nozzle ramp, and the slope of the ramped groove are approximately equal.

27. The lancing device of claim 26, wherein the detent forms a cantilevered portion of the collar ramp.

28. The lancing device of claim 26, wherein the detent forms a slotted portion of the collar ramp.

29. The method of claim 24, wherein the raised boss can oppose the collar pin sliding from the ramped groove to the assembly groove.

30. The method of claim 24, wherein the ramped groove comprises an over-rotation groove.

31. The method of claim 24, wherein the collar further comprises one or more collar alignment features, the method further comprising:
providing an exterior nozzle comprising one or more exterior nozzle alignment features that can engage the one or more collar alignment features;
aligning the one or more collar alignment features with the one or more exterior nozzle alignment features; and
engaging the exterior nozzle to the collar.

32. A method of adjusting a lancing depth of a nozzle assembly in a lancing device, the nozzle assembly comprising an exterior nozzle, an interior nozzle comprising a ramped groove, and a collar engaging the exterior nozzle and comprising a collar pin that can slidably engage the ramped groove, the method comprising:
rotating the exterior nozzle and the collar relative to the interior nozzle to slide the collar pin in the ramped groove of the interior nozzle to adjust the lancing depth.

33. The method of claim 32, wherein the collar further comprises a collar ramp, the collar ramp comprising a detent, and the interior nozzle further comprises an interior nozzle ramp, the interior nozzle ramp comprising a plurality of adjustment notches that can engage the detent, and wherein rotating the exterior nozzle and the collar relative to the interior nozzle to slide the collar pin in the ramped groove of the interior nozzle to adjust the lancing depth further comprises:
selecting the lancing depth by rotating the exterior nozzle to engage the detent with one of the adjustment notches.

34. The method of claim 33, wherein the slope of the collar ramp, the slope of the interior nozzle ramp, and the slope of the ramped groove are approximately equal.

35. The method of claim 33, wherein the detent forms a cantilevered portion of the collar ramp.

36. The method of claim 33, wherein the detent forms a slotted portion of the collar ramp.

37. The method of claim 33, wherein the ramped groove comprises an over-rotation groove.
